(19) United States
(12) Patent Application Publication
(10) Pub. No.: US 2017/0001030 A1
(43) Pub. Date: Jan. 5, 2017

(54) MAGNETIC STIMULATION DEVICE AND METHODS

(71) Applicant: BTL HOLDINGS LIMITED, Nicosia (CY)

(72) Inventors: Ondrej Pribula, Praha (CZ); Tomás Schwarz, Praha (CZ)

(21) Appl. No.: 15/178,455

(22) Filed: Jun. 9, 2016

Related U.S. Application Data
(63) Continuation-in-part of application No. 15/073,318, filed on Mar. 17, 2016.
Application No. 15/073,318 is a continuation-in-part of application No. 14/951,093, filed on Nov. 24, 2015.
Application No. 14/951,093 is a continuation-in-part of application No. 14/926,365, filed on Oct. 29, 2015.
Application No. 14/926,365 is a continuation-in-part of application No. 14/789,658, filed on Jul. 1, 2015.
Application No. 14/789,658 is a continuation-in-part of application No. 14/873,110, filed on Oct. 1, 2015.
Application No. 14/873,110 is a continuation of application No. 14/789,156, filed on Jul. 1, 2015, now abandoned.
Application No. 14/789,156 is a continuation-in-part of application No. 15/099,274, filed on Apr. 14, 2016.
Application No. 15/099,274 is a continuation-in-part of application No. 15/151,012, filed on May 10, 2016.

(51) Int. Cl. A61N 2/12; A61N 2/00; A61M 19/00; H01F 7/06

(52) U.S. Cl. CPC A61N 2/12; H01F 7/064; A61N 2/008; A61M 19/00; A61N 2/006

(57) ABSTRACT

A magnetic stimulation device and methods use a plurality of magnetic field generating devices, allowing for faster treatment of a patient and for treatment of large areas of the body. The shape of the generated magnetic field may be adjusted by an operator. The magnetic stimulation device is able to provide a plurality of various treatments at the same time.

### PRIORITY CLAIM

This application is a Continuation-in-Part of each of the following: U.S. patent application No. 15/073,318 filed Mar. 17, 2016 and now pending; No. 14/951,093 filed Nov. 24, 2015 and now pending; No. 14/926,365 filed Oct. 29, 2015 and now pending; and No. 14/789,658 filed Jul. 1, 2015 and now pending. This application is also a Continuation-in-Part of U.S. patent application Ser. No. 14/873,110 filed Oct. 1, 2015 and now pending, which is a Continuation of U.S. patent application Ser. No. 14/789,156 filed Jul. 1, 2015 and now abandoned. This application is also a Continuation-in-Part of U.S. patent application Ser. No. 15/099,274 filed Apr. 14, 2016 and now pending. This application is also a Continuation-in-Part of U.S. patent application Ser. No. 15/151,012 filed May 10, 2016 and now pending.

### FIELD OF THE INVENTION

The present invention relates to apparatus and methods for treating a patient by a magnetic field. The application of the magnetic field is provided by a plurality of magnetic field generating devices.

### BACKGROUND OF THE INVENTION

Devices and methods generating magnetic pulses have been used for medical and/or aesthetic treatments. A time-varying magnetic field induces electric currents in the patient's body, which may provide an effect similar to electrotherapeutic treatment. The induced electrical currents may evoke an active response by sufficient intensity, impulse duration and/or repetition rate.

The currently used magnetic stimulation devices mostly consist of one magnetic field generating device, a capacitor parallel to a power source and a switching device in series to the power source. Further such topology requires a snubbering device, such as diode or RC snubbering circuit, for protecting the energy source during the reverse polarity of resonance. The use of a snubbering device causes high energy losses. The magnetic stimulation device wastes a lot of energy because of low efficiency due to significant electric losses while generating the time-varying magnetic field. Therefore these devices generate magnetic impulses ineffectively.

Further there is a need to treat different areas by magnetic field to provide more efficient and faster treatment of large areas. The large areas may be at least one muscle group or region prone to cellulite.

Another disadvantage of currently used devices using one magnetic field generating device is the shape of the magnetic field. The shape of the magnetic field is dependent on the shape of the magnetic field generating device. The magnetic field may not be selectively focused during the treatment. The adjustable parameters are magnetic flux density, repetition rate and/or time duration of the treatment.

Still another disadvantage of currently used magnetic stimulation devices is the slow treatment in the case of large and/or a plurality of treated areas. The applicator has to be manually moved from one position to another position to stimulate the large and/or the plurality of treated areas and the treatment of the entire area takes a long time. Further the treatment is not homogenous since the magnetic field does not stimulate the entire large target area at the same time.

Furthermore magnetic stimulation devices using a plurality of magnetic field generating devices have limited effectiveness due to the serial connection of the switching device and magnetic field generating device which requires a snubbering device, causing high energy losses.

### SUMMARY OF THE INVENTION

The present invention provides a magnetic stimulation device and method of controlling the magnetic stimulation device using a plurality of magnetic field generating devices.

Using a plurality of magnetic field generating devices provides faster treatment. Large and/or different areas may be treated in shorter time. Using a plurality of applicators allows different areas and/or target biological structures to be stimulated at the same time. The movement of the at least one applicator may automatically follow a predetermined trajectory. Hence manual manipulation is not needed. Furthermore the shape of the generated magnetic field may be adjusted by an operator.

The treatment effect is more focused since the magnetic stimulation may be focused by interference of the magnetic fields generated by the plurality of magnetic field generating devices. The magnetic field may selectively stimulate small biological structures and the treatment effect may be more effective. The results may be achieved faster since the treatment is enhanced due to using the plurality of magnetic field generating devices.

The treatment is more effective with reference to the currently used magnetic stimulation devices due to use of a parallel switching device with reference to the energy source, since the effectivity is higher due to lower energy losses. Additionally there are no losses in snubbering devices no snubbering device is needed or used. Additionally the hardware components may be rated at the nominal working voltage of the power supply even during resonance. This provides a significant advantage relative to existing devices which use serial switch topology where during resonance the hardware components are loaded by a voltage approximately double the nominal working voltage.

### BRIEF DESCRIPTION OF THE DRAWINGS

FIG. 1 illustrates an operation mode when impulses are generated by the plurality of magnetic field generating devices at one time within a pulse.

FIG. 2 illustrates an operation mode when impulses are generated by the plurality of magnetic field generating devices at plurality of different times within a pulse.

FIG. 3 illustrates an exemplary embodiment of a magnetic stimulation device including a plurality of magnetic field generating devices generating time-dependent impulses.

FIG. 4 illustrates an exemplary embodiment of a magnetic stimulation device including a plurality of magnetic field generating devices generating time-independent impulses.

FIG. 5 illustrates an exemplary embodiment of a magnetic stimulation device including a plurality of magnetic field generating devices generating time-independent impulses.

### LIST OF REFERENCE NUMBERS

1 impulse
2 pulse
3 energy source
4 energy storage device
5-7 magnetic field generating device
8-11 switching device
12-14 energy storage device
15-17 magnetic field generating device
18-23 switching device
24 energy storage device
25 magnetic field generating device
26 switching device

### GLOSSARY

Impulse refers to a single magnetic stimulus.

Pulse refers to a period of stimulation signal consisting of at least one biphasic stimulus and a time duration of no stimulation, i.e. time duration between two impulses from a first edge rise to a next edge rise.

Treatment parameters refer to magnetic flux density, repetition rate, impulse duration or treatment duration.

### DETAILED DESCRIPTION

Most of the currently used magnet treatment devices includes the only one magnetic field generating device for treatment of a patient. An applicator including one magnetic field generating device is focused for treatment of the particular target area of the patient.

However, there are often applications where the target area is larger than the area one magnetic field generating device is able to stimulate, e.g. at least one muscle group, such as quads, glutes or pelvic floor muscles, abs, biceps and/or triceps or any region prone to cellulite, such as a belly region, love handles, buttocks, thigh region etc. In the currently used treatment devices these large target areas are treated by moving and repositioning the magnetic field generating device which leads to slow treatment.

In one aspect of the invention a magnetic stimulation device includes at least one applicator and a plurality of magnetic field generating devices. The magnetic field generating device may be air-cooled or cooled by any other fluid media.

The magnetic stimulation device may include at least one energy source, at least one energy storage device (e.g. a capacitor), at least one magnetic field generating device (e.g. a coil) and at least one switching device. The magnetic field generating device may include a core, however in a preferred embodiment the magnetic field generating device includes no core. The switching device may be any kind of switch such as diode, MOSFET, JFET, IGBT, BJT, thyristor or a combination of them.

The at least one magnetic generating device may be in various shapes to enhance a variability of magnetic field profile. The shape of the magnetic field generating device may be circular, semicircular, rectangular, “figure 8”, V-shape, Y-shape or a butterfly shape. The magnetic field generating device may be flat (2-D shape). In an alternative embodiment the magnetic field generating device may correspond to various 3-D bodies, e.g. a hemisphere. In another alternative embodiment the magnetic field generating device may be flexible to be better fitted to the patient. The magnetic field generating device may or may not include a core for the field shaping.

The positioning of the plurality of magnetic generating device may be very useful.

Large areas may be stimulated by the plurality of the magnetic field generating devices. The plurality of magnetic field generating devices may generate a plurality of independent magnetic fields, e.g. two magnetic field generating devices may generate two magnetic fields with two peaks of magnitude of magnetic flux density.

The plurality of magnetic field generating devices may be operated at various treatment parameters and/or operation modes to provide various treatment effects for the patient during the treatment, e.g. myostimulation, myorelaxation, analgesic effect or aesthetic effects such as adipose tissue reduction, muscle toning, muscle shaping, body contouring, body shaping, skin tightening, cellulite treatment, circumferential reduction, breast enhancement and/or lip enhancement.

The magnetic field generating devices may be positioned in isolated locations of the applicator. Alternatively, the magnetic field generating devices may be positioned next to each other, in an array or matrix, in a pattern or in randomized locations of the applicator.

The magnetic field generating devices may be positioned and/or movable in the at least one applicator in one plane; in at least two mutually tilted planes defined by a convex or concave angle, or perpendicular to each other; or in at least two parallel planes with the at least one magnetic field generating device in each parallel plane. The movement of the at least one magnetic field generating device may be translational and/or rotational, constant or accelerated. The movement may follow a predetermined, random or predefined trajectory, such as a pattern, array or matrix. The angles of the planes and/or the movement of the at least one magnetic field generating device may be adjusted by an operator following the patient's needs. In an alternative embodiment the patient may be positioned in the intersection of the magnetic fields generated by the plurality of magnetic field generating devices.

The positions of the at least two magnetic field generating devices may focus the magnetic fields to the target area; or the magnetic field generated by one magnetic field generating device may interfere with the magnetic field generated by another magnetic field generating device and the resulting magnetic field may be shaped. The magnetic flux density may be summed from the plurality of magnetic field generating devices.

The plurality of the magnetic field generating devices may extend the active time duration of the stimulation in the case that the switching devices are switched in sequence. Therefore the treatment is more effective and the treatment time may be shortened.

The magnetic stimulation device may include the at least one applicator, the at least one energy source and at least two magnetic field generating devices. However, in an alternative embodiment the magnetic stimulation device may include a plurality of applicators and/or plurality of energy sources. The plurality of applicators may be used for treatment of at least two cooperating muscle groups with different treatment effects. In an exemplary application e.g. the triceps brachii muscle may be treated to achieve myostimulation effects and the biceps brachii muscle may be treated to achieve myorelaxation effects.

The at least one applicator may be movable in a predefined pattern, e.g. a scanning movement, or the movement may follow a random trajectory. The movement may be constant or accelerated to provide the most comfortable treatment for the patient. The movement of the applicator may be adjusted by the operator.

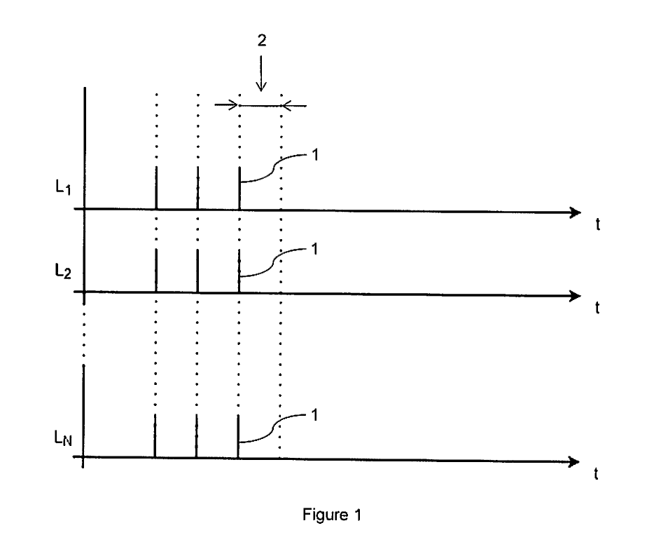

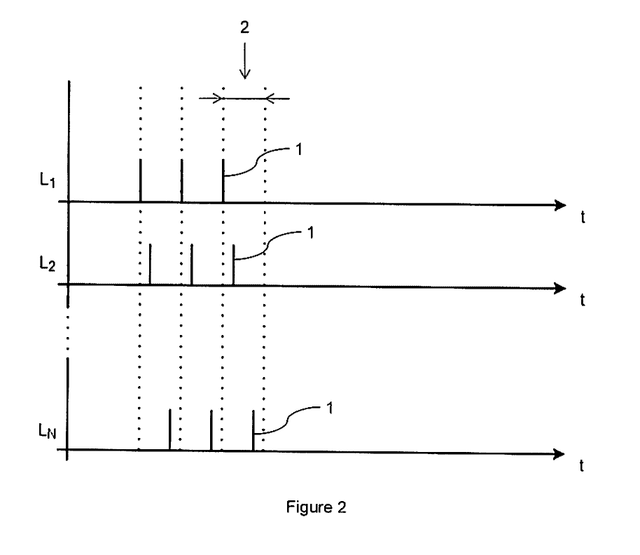

The treatment by the magnetic stimulation device may be in different operation modes. One operation mode may generate a plurality of impulses 1 at one time within the pulse 2 as illustrated in FIG. 1. Another operation mode may generate a plurality of the impulses 1 at different times within the pulse 2 as illustrated in FIG. 2.

Both operation modes may be combined.

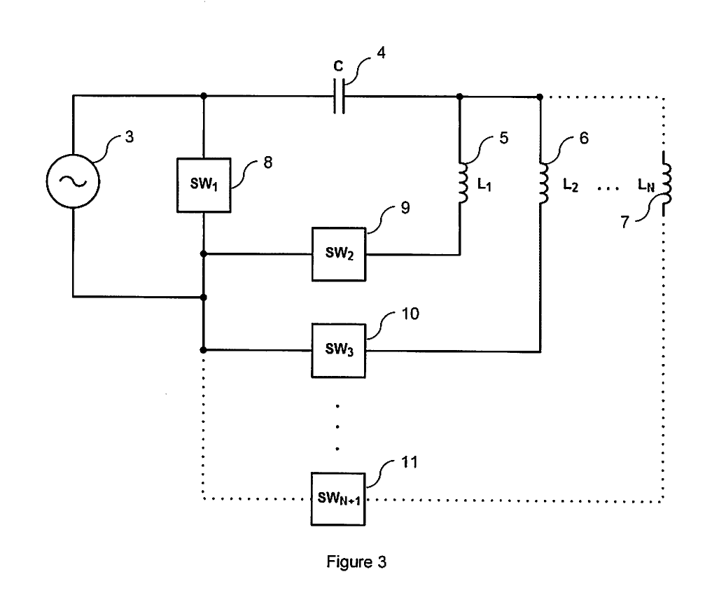

The magnetic stimulation device may generate a plurality of the impulses 1 by the magnetic field generation devices L₁, L₂, . . . L_{N} at one time within the pulse 2. This operation mode is illustrated in FIG. 1. As shown in FIG. 3, a magnetic stimulation device may include at least one energy source 3, one energy storage device 4, N magnetic field generating devices 5-7 and N+1 switching devices 8-11, wherein N is positive integer greater than 1. This exemplary embodiment includes a minimum of hardware components. The value of inductance of each magnetic field generating device may be constant, however in an alternative embodiment different values of inductance may be used.

The switching devices 9-11 may be switched separately, with the magnetic field generated by separate magnetic field generating devices.

In an alternative embodiment any switching device may be switched in combination with at least one other switching device.

The magnetic flux density of the stimulation is proportional to the number and/or the inductance of active magnetic field generating devices 9-11. The active magnetic field generating devices are the magnetic field generating device in the closed loop of the electric circuit. For example if the number of active magnetic field generating devices is 2 and the inductances of the magnetic field generating devices are the same, then the value of magnetic flux density for each magnetic field generating device is one-half of the magnetic flux density which would be reached by one active magnetic field generating device with the same parameters and conditions, e.g. inductance, resistance, frequency, voltage. The total equivalent inductance of the magnetic stimulation device may be changed by switching a plurality of switching devices into a closed electric circuit. Therefore the impulse duration may be adjusted by adjusting the inductance. The value of total equivalent inductance (Lₜₒₜₐₗ) may be determined by Equation 1.

$$\frac{1}{{L}_{total}} = \frac{1}{{L}_{1}} + \frac{1}{{L}_{2}} + … + \frac{1}{{L}_{N}} \qquad Equation 1$$

The magnetic stimulation device may generate a plurality of impulses 1 generated by the magnetic field generation devices L₁, L₂, . . . L_{N} at different times within the pulse 2. This operation mode is illustrated in FIG. 2. This operation mode may multiply the repetition rate perceived by the patient, e.g. when the number of magnetic field generation device is 3 and the repetition rate of each magnetic field generating device is 100 Hz, then the patient may perceive the repetition rate 300 Hz. In an alternative example, this operation mode may be used for treatments of high repetition rate when the magnetic stimulation devices are switched to reach such repetition rates which may be sufficiently cooled.

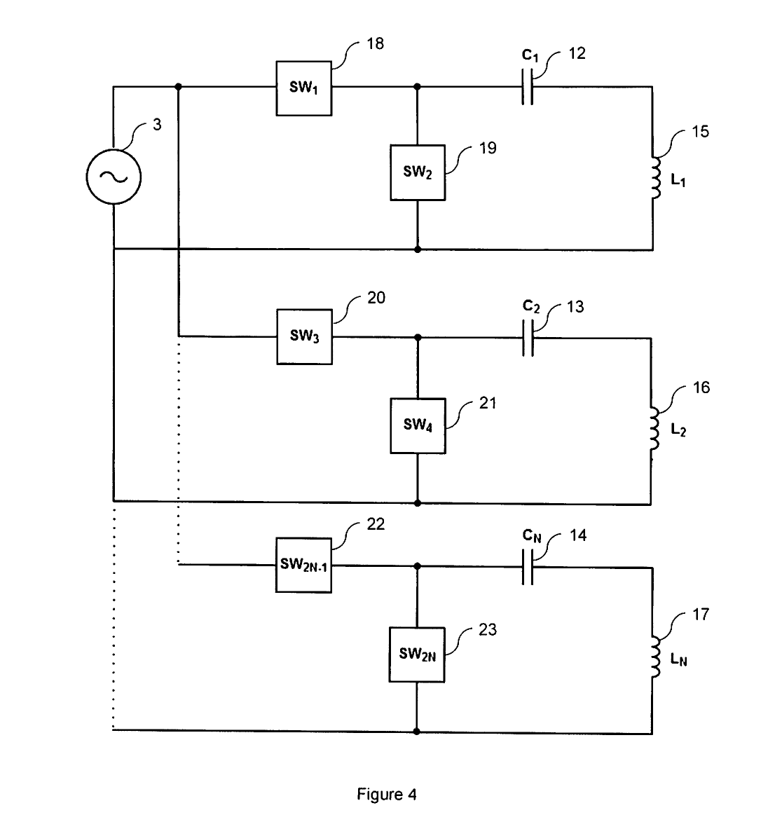

In the example of FIG. 4 a magnetic stimulation device includes at least one energy source 3, N energy storage devices 12-14, N magnetic field generating devices 15-17 and 2×N switching devices 18-23, wherein N is positive integer greater than 1. The at least one energy storage device 12-14 may be selectively charged by the energy source 3 by selectively switching the switching devices 18, 20, 22 and the impulses may be selectively generated by selectively switching the switching devices 19, 21, 23.

The benefit of this exemplary embodiment is the time independency of the impulses generated by the separate magnetic field generating devices. However, the switching devices may be synchronized to generate the impulses at one fixed time within the pulse or both operation modes may be combined using this embodiment. Another benefit of this embodiment is the possibility of providing various treatments by a plurality of magnetic field generating devices. Various treatments may provide various effects for the patient, e.g. stimulation, such as myostimulation, pain alleviation or myorelaxation.

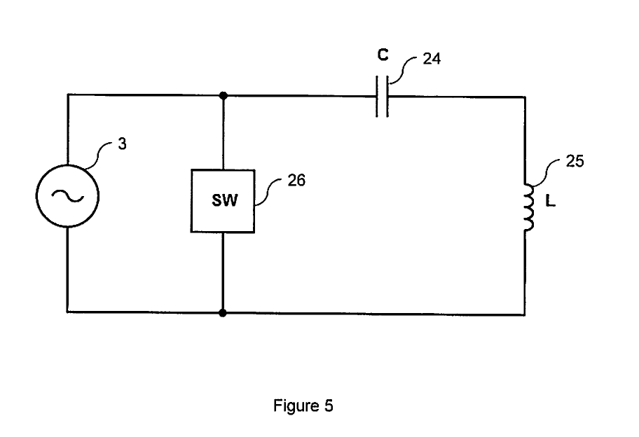

FIG. 5 illustrates an example where the magnetic stimulation device includes N energy sources 3, N energy storage device 24, N magnetic field generating devices 25 and N switching devices 26, wherein N is positive integer greater than 1. The at least one energy storage device 24 may be selectively charged by the energy source 3 and the impulses may be selectively generated by selectively switching the switching devices 26.

The impulses generated by the separate magnetic field generating devices are time independent. However, the switching devices may be synchronized to generate the impulses at one time within the pulse or both operation modes may be combined.

The magnetic stimulation device may include a plurality of applicators. The applicator includes at least one magnetic field generating device which may be movable. The benefit of this embodiment is that the movement and/or positioning of the plurality of the applicators may be independent. Hence different parts of the patient's body may be treated simultaneously. Therefore the total treatment time is reduced and patient's downtimes are reduced as well. The movement of the at least one applicator may be automatic so that manual manipulation may not be needed. The movement of the at least one applicator may follow a predetermined trajectory or it may be random. In an alternative embodiment the movement of the plurality of applicators may be synchronized.

The plurality of applicators may be positioned with respect to each other in one plane; in at least two mutually tilted planes defined by convex or concave angles, or perpendicular to each other; or in at least two parallel planes. The angles of the planes may be adjusted by an operator following the patient's needs. In an alternative embodiment the patient may be positioned in the intersection of the magnetic fields generated by the plurality of magnetic field generating devices.

The benefit of this application may be treatment of a plurality of cooperating muscles, such as agonists and antagonists, e.g. one muscle may be stimulated to achieve strengthening effect and on the other side the other muscle may be stimulated to achieve myorelaxation effect.

All the above described exemplary embodiments may be used in one or a plurality of applicators.

The inductance of magnetic field generating devices in each embodiment may vary. The capacitance of the energy storage devices in each embodiment may vary as well. The impulse duration may be variable and/or the magnetic flux density generated by different magnetic field generating devices may vary as well.

Thus, novel devices and methods have been shown and described. Various changes and substitutions may of course be made without departing from the spirit and scope of the invention. The invention, therefore, should not be limited, except to the following claims and their equivalents.

1. A magnetic stimulation device including:
at least one applicator, at least one energy source, a plurality of switching devices, at least one energy storage device and a plurality of magnetic field generating devices, wherein the at least one switching device is electrically connected in parallel to the energy source.

2. The device of claim 1, wherein the plurality of magnetic field generating devices are positioned with respect to each other.

3. The device of claim 2, wherein the plurality of magnetic field generating devices are in one plane.

4. The device of claim 2, wherein the plurality of magnetic field generating devices are in a plurality of planes.

5. The device of claim 4, wherein at least one plane is tilted and/or parallel with respect to the at least one another plane.

6. The device of claim 1, wherein the magnetic stimulation device includes a plurality of applicators.

7. The device of claim 6, wherein the at least one magnetic field generating devices is in the at least one applicator.

8. The device of claim 1, wherein the magnetic stimulation device includes at least one applicator, one energy source, one energy storage device, N magnetic field generating devices and N+1, wherein N is positive integer greater than 1.

9. The device of claim 8, wherein the value of inductance of at least one magnetic field generating device differs from the value of inductance of at least one another magnetic field generating device.

10. The method of claim 8, further including adjusting total equivalent inductance of the magnetic stimulation device based on the number of active magnetic field generating devices used.

11. A method of operating a magnetic stimulation device including at least one applicator, at least one energy source, a plurality of switching devices, at least one energy storage device and a plurality of magnetic field generating devices, comprising moving the at least one magnetic field generating device.

12. The method of claim 11, wherein the movement of the at least one magnetic field generating device is constant or accelerated.

13. The method of claim 11, wherein the movement of the at least one magnetic field generating device is translational and/or rotational.

14. The method of claim 11, wherein the movement of the at least one magnetic field generating device is random.

15. The method of claim 11, wherein the movement of the at least one magnetic field generating device follows a predetermined trajectory.

16. The method of claim 15, wherein the predetermined trajectory is a pattern, an array or a matrix.

17. A method of operating a magnetic stimulation device including at least one applicator, at least one energy source, a plurality of switching devices, a plurality of energy storage devices and first and second magnetic field generating devices comprising:
generating impulses via the first magnetic field generating device; and
generating impulses via the second magnetic field generating device independently of the impulses generated by the first magnetic field generating device.

18. The method of claim 17, wherein the energy storage devices are recharged by the at least one energy source simultaneously or at different times within one pulse.

19. The method of claim 17, wherein the first magnetic field generating device generates a first magnetic field of different treatment parameters from the a second magnetic field generated by the second magnetic field generating device.

20. The method of claim 17, wherein the first and second magnetic field generation devices generates impulses at one time within one pulse.

21. The method of claim 20, wherein the first and second magnetic fields interfere.

22. The method of claim 17, wherein the first and second magnetic field generation devices generate impulses in a plurality of different times within one pulse.

23. The method of claim 17, wherein myostimulation, myorelaxation, analgesic effect or at least one aesthetic effect is achieved.

24. The method of claim 17 further comprising positioning a treatment target area of a patient at an intersection of the magnetic fields generated by the plurality of magnetic field generating devices.

25. The method of claim 17, wherein the magnetic stimulation device includes at least one applicator, one energy source, N energy storage devices, N magnetic field generating devices and 2N switching devices, wherein N is positive integer greater than 1.

26. The method of claim 17, wherein the magnetic stimulation device includes at least one applicator, N energy sources, N energy storage devices, N magnetic field generating devices and N switching devices, wherein N is positive integer greater than 1.

27. A method of operating a magnetic stimulation device including at least one applicator, at least one energy source, a plurality of switching devices, at least one energy storage device and a plurality of magnetic field generating devices, comprising:
generating a plurality of peaks of magnitudes of magnetic flux density using a plurality of magnetic field generating devices.

28. The method of claim 27, wherein the plurality of magnetic field generating devices treats a large area.
